(19) United States
(12) Patent Application Publication
(10) Pub. No.: US 2003/0156256 A1
(43) Pub. Date: Aug. 21, 2003

(54) Eye's optical characteristic measuring system

(76) Inventors: Gaku Takeuchi, Tokyo-to (JP); Katsuhiko Kobayashi, Tokyo-to (JP); Masahiro Shibutani, Tokyo-to (JP); Yumi Kubotera, Tokyo-to (JP)

Correspondence Address: Kevin S. Lemack, Nields & Lemack, Suite 7, 176 E. Main Street, Westboro, MA, 01581, US

(21) Appl. No.: 10/364,771

(22) Filed: Feb. 11, 2003

(30) Foreign Application Priority Data
Feb. 15, 2002 (JP) 2002-39154

(51) Int. Cl. A61B 3/10

(52) U.S. Cl. 351/205

(57) ABSTRACT

An eye's optical characteristic measuring system, comprising a target projecting system for projecting a target image on an ocular fundus of an eye under test, a photodetection optical system for guiding the target image to a photoelectric detector, a calculating unit for calculating an optical transmission function of the eye under test based on light amount intensity distribution of the target image detected by the photoelectric detector, and an arithmetic unit for calculating visual acuity of a person under test from intersection of the optical transmission function and a predetermined threshold value.

### BACKGROUND OF THE INVENTION

The present invention relates to a eye's optical characteristic measuring system capable of estimating and calculating a visual acuity of an eye under test based on light amount intensity distribution characteristic of a target image projected on a fundus of the eye.

In the past, the present applicant has already filed a patent application for a system, which comprises a target projecting system for projecting a target image to an ocular fundus of an eye under test, and a photodetection optical system for guiding the target image to a photoelectric detector. Based on light amount intensity distribution of the target image detected by the photoelectric detector, the system calculates a simulation image on the fundus, which would be formed when the target image is projected to the fundus of the eye. Then, the system can identify what kind of image is formed on the fundus of the eye under test.

The system as described above provides an effect such that it is possible to calculate and identify in what condition various types of target images are projected to the fundus of the eye without actually projecting the various types of target images.

However, in the above system already in application, an image itself obtained by simulation can be observed, while, with respect to the visual acuity value, a visual acuity value of the eye under test must be estimated by the tester himself based on the result of observation. In this respect, there has been problem that it is difficult to find accurate visual acuity value.

### SUMMARY OF THE INVENTION

To solve the above problems of the conventional type eye's optical characteristic measuring system used in the past, it is an object of the present invention to provide a system, by which it is possible to obtain an accurate visual acuity value objectively from measurement data without asking the result of the observation to a person under test.

To attain the above object, the eye's optical characteristic measuring system according to the present invention comprises a target projecting system for projecting a target image on an ocular fundus of an eye under test, a photodetection optical system for guiding the target image to a photoelectric detector, a calculating unit for calculating an optical transmission function of the eye under test based on light amount intensity distribution of the target image detected by the photoelectric detector, and an arithmetic unit for calculating visual acuity of a person under test from intersection of the optical transmission function and a predetermined threshold value. Also, the present invention provides an eye's optical characteristic measuring system as described above, wherein the optical transmission function is square wave frequency characteristics. Further, the present invention provides an eye's optical characteristic measuring system as described above, wherein the threshold value is a modulation threshold. Also, the present invention provides an eye's optical characteristic measuring system as described above, wherein a plurality of the modulation threshold are prepared to correspond to age of the persons under test, and a modulation threshold suitable for each person under test is used.

### BRIEF DESCRIPTION OF THE DRAWINGS

FIG. 1 is a schematical drawing of an ocular fundus of a human eye;

FIG. 2 is a basic block diagram of an eye's optical characteristic measuring system according to an embodiment of the present invention;

FIG. 3(A) and FIG. 3(B) each represents a drawing to show a condition of reflection at an ocular fundus of an eye under test in the eye's optical characteristic measuring system; and

FIG. 4 is a diagram showing relation between optical transmission function of en eye under test and a threshold value corresponding to each age group.

### DETAILED DESCRIPTION OF THE PREFERRED EMBODIMENT

Description will be given below on an embodiment of the present invention referring to the drawings.

First, brief description will be given on tissues of an ocular fundus of a human eye.

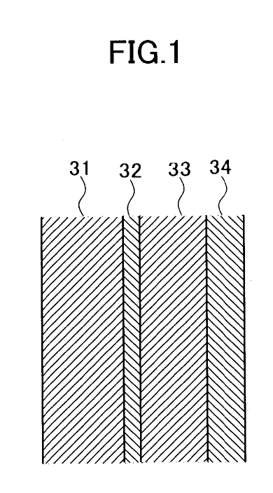

FIG. 1 is a schematical drawing of tissues of an ocular fundus of a human eye. Reference numeral 31 denotes a visual cell layer, 32 is a retinal pigment epithelial layer, 33 is a choroidal membrane, and 34 is a sclera.

The visual cell layer 31 is an aggregation of fibrous visual cells aligned perpendicularly to the retinal pigment epithelial layer 32. A light beam passing through the visual cell layer 31 (visual cell) is reflected with mirror reflection by the retinal pigment epithelial layer 32. On the other hand, a part of the light beam passes through the retinal pigment epithelial layer 32 and is reflected with scattering reflection by the choroidal membrane 33 and the sclera 34 positioned behind. However, the light reflected by scattering reflection exerts almost no influence on an image to be observed and recognized by a person.

It is demonstrated in the experiment that, when the light beam entering the visual cell layer 31 passes through the visual cell, the light beam passes through it by repeating the reflection almost similar to total reflection in the visual cell.

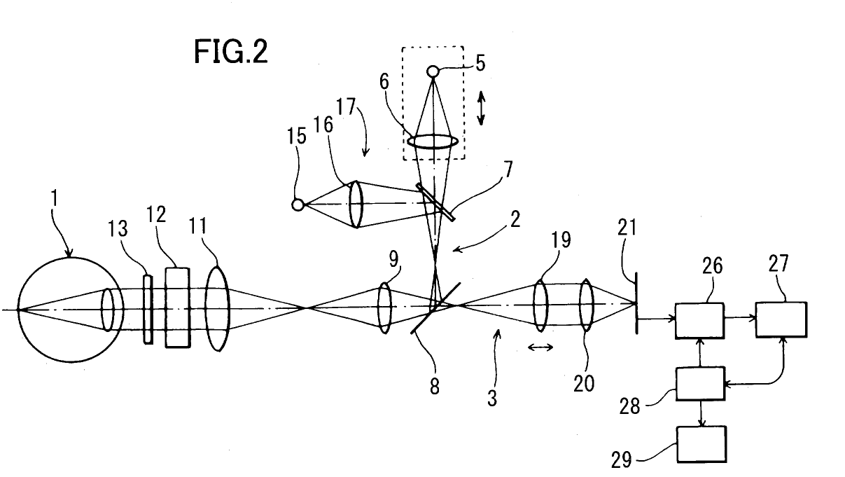

FIG. 2 shows a basic block diagram of an eye's optical characteristic measuring system according to an embodiment of the present invention.

In this figure, reference numeral 1 is an eye under test, 2 is a target projecting optical system for projecting a target image, and 3 is a photodetection optical system for receiving the light beam reflected from the eye under test.

The projecting optical system 2 comprises a light source 5, a projection lens 6 for converging a projected light beam emitted from the light source 5, a half-mirror 7 arranged on an optical axis of the projection lens 6, a polarization beam splitter 8 for directing the projected light beam passing through the half-mirror 7, for reflecting and projecting a linear polarization component (P linearly polarized light) in a first direction of polarization toward the eye 1 under test and for allowing S linearly polarized light having a direction of polarization deviated by 90° from P linearly polarized light to pass, a relay lens 9 arranged on a projection optical axis of the polarization beam splitter 8 closer to the polarization beam splitter 8 side, an objective lens 11, a correction optical system 12 arranged between the objective lens 11 and the eye 1 under test and comprising a spherical lens, and a ¼ wave plate 13. Further, a gaze target system 17 is arranged to face to the half-mirror 7 and comprises a gaze target 15 and a condenser lens 16. The light source 5 and the gaze target 15 are placed at positions conjugate to the ocular fundus of the eye 1 under test. As to be described later, the light source 5 and the gaze target 15 form an image on the ocular fundus. The light source 5 is integrated with the projection lens 6, and these can be moved in direction of the optical axis in linkage with a focusing lens 19 (to be described later).

The photodetection optical system 3 shares the following components with the projecting optical system 2: the polarization beam splitter 8, the relay lens 9 arranged on the projection optical axis of the polarization beam splitter 8, the objective lens 11, the correction optical system 12, and the ¼ wave plate.

On an optical axis of the reflected light passing through the polarization beam splitter 8, there are provided the focusing lens 19 movable along the reflection light optical axis and an image forming lens 20. The image forming lens 20 focus the reflection light beam on a photoelectric detector 21, which is arranged at a position conjugate to the ocular fundus of the eye 1 under test.

A photodetection signal from the photoelectric detector 21 is stored in a storage unit 27 via a signal processing unit 26. The writing of data from the signal processing unit 26 to the storage unit 27 is controlled by a control unit 28. The control unit 28 has a visual acuity calculating unit and calculates an estimated visual acuity value by required calculation based on the data stored in the storage unit 27. The result of the calculation is displayed on a display unit 29.

Now, description will be given on operation of the optical system.

The focusing lens 19 is positioned at a reference position, and a person with the eye 1 under test is instructed to gaze at the gaze target 15. In this case, the correction optical system 12 is set to a correction amount 0.

With the eye 1 gazing at the gaze target 15, a projecting light beam is projected to the ocular fundus of the eye 1 by the projecting optical system 2, and an image of a point light source is formed on the ocular fundus of the eye 1 under test. Visual light is used for the gaze target 15, and infrared light is used for the projected light beam.

The projected light beam (infrared light) from the light source 5 passes through the projection lens 6 and the half-mirror 7 and reaches the polarization beam splitter 8. At the polarization beam splitter 8, a P linearly polarized light component is reflected. This passes through the relay lens 9, and is projected to the ocular fundus of the eye 1 under test by the objective lens 11, and the correction optical system 12 via the ¼ wave plate 13, and a first target image is formed on the ocular fundus.

When the P linearly polarized light passes through the ¼ wave plate 13, it is turned to a right circularly polarized light. The projected light beam is totally reflected by the ocular fundus of the eye 1, and the totally reflected light beam is turned to a left circularly polarized light when it is reflected by the ocular fundus. Further, when the totally reflected light beam passes through the ¼ wave plate 13, it is turned to an S linearly polarized light, which has a direction of polarization deviated by 90° from a direction of polarization of the P linearly polarized light.

The S linearly polarized light is guided to the polarization beam splitter 8 via the correction optical system 12, the objective lens 11 and the relay lens 9. The polarization beam splitter 8 reflects the P linearly polarized light and allows the S linearly polarized light to pass. Thus, the totally reflected light beam passes through the polarization beam splitter 8 and forms an image as a second target image on the photoelectric detector 21 by the focusing lens 19 and the image forming lens 20.

Incidentally, the projected light beam projected to the ocular fundus of the eye 1 under test is not totally reflected by a surface of the fundus with mirror reflection. A part of the light beam enters into a superficial layer through the surface of the fundus and is reflected with scattering reflection, i.e. the so-called bleeding reflection occurs. When the light beam reflected with scattering reflection is received by the photoelectric detector 21 at the same time as the light beam reflected with mirror reflection, it is turned to noise in light amount intensity distribution of the second target image, and the eye's optical characteristic of the optical system of the eye cannot be accurately measured.

The condition of polarization of the light beam reflected by scattering reflection is in random status. For this reason, when the light beam passes through the ¼ wave plate 13 and is turned to a linearly polarized light, the component matching with the S linearly polarized light is restricted to a limited part. The components other than the components matching with the S linearly polarized light in the light beam reflected by scattering light are reflected by the polarization beam splitter 8. Therefore, the ratio of A to B is negligibly low, where A is the S linearly polarized light component of the light beam reflected with scattering reflection and B is the S linearly polarized light component reflected with mirror reflection at the ocular fundus of the eye 1.

Accordingly, the light received by the photoelectric detector 21 is the reflected light beam with mirror reflection, which substantially removes the reflected light component with scattering reflection. By adding the ¼ wave plate 13 as a component element of the projecting optical system 2 and the photodetection optical system 3, eye's optical characteristic of the optical system of the eye can be accurately measured. The control unit 28 calculates the light amount intensity distribution characteristic, and an optical transmission function of the optical system of the eye based on a photodetection signal from the photoelectric detector 21 and also on the data stored in the storage unit 27. Further, the estimated visual acuity value of the eye 1 under test is calculated according to the optical transmission function.

The optical characteristic of the ocular fundus can be measured by the following procedure:

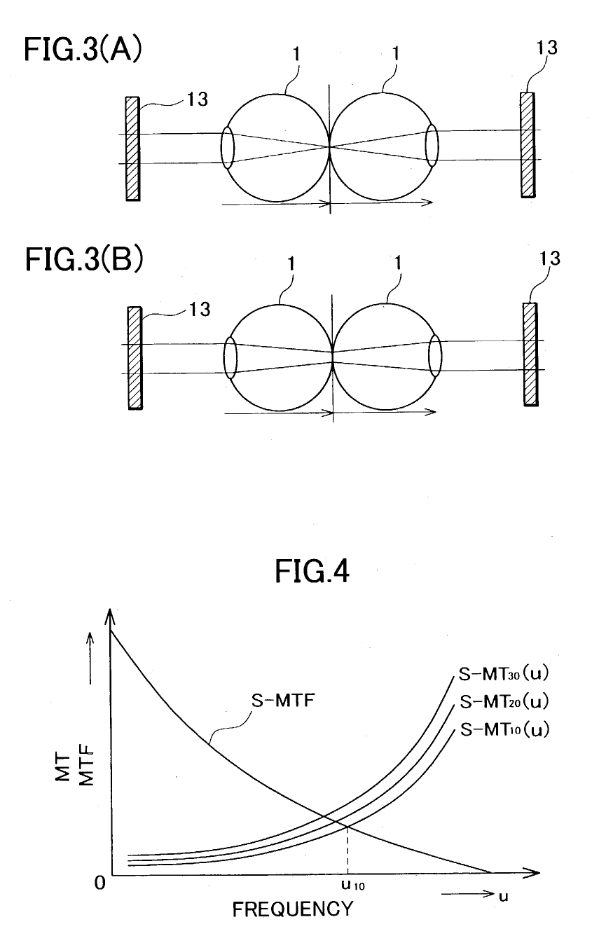

FIG. 3(A) shows a condition when the light beam is focused on the ocular fundus, and FIG. 3(B) shows a condition when the light beam is not focused on the ocular fundus. Because of the influence of the detailed structure of the ocular fundus as described above, the following relationship exists under both conditions:

P(x,y)※ R(x,y)※ R(x,y)※ P(x,y)=I(x,y) (1)

where P (x,y) is amplitude transmittance of the eye's optical system of the eye 1 under test, R (x,y) is amplitude transmittance of the visual cells including reflection characteristics at the retinal pigment epithelial layer 32, and I (x,y) is 2-demensional light amount intensity distribution to be measured on a 2-dimensional detector calculated from the photodetection signal from the 2-dimensional detector (photoelectric detector 21).

The mark ※ indicates convolution integration.

Next, both sides of the equation (1) are processed by Fourier transform.

Here, if it is assumed that p (u,v) is an optical transmission function of the optical system of the eye, r (u,v) is an optical transmission function of the visual cell, and i (u,v) is a 2-dimensional optical transmission function on the 2-dimensional detector, the following relationship exists:

FT[P(x,y)]=p(u,v)

FT[R(x,y)]=r(u,v)

FT[I(x,y)]=i(u,v)

By Fourier transform of the equation (1):

p(u,v)×[r(u,v)]²×p(u,v)=i(u,v) (2)

Therefore, the following equation is approximately established:

[p(u,v)r(u,v)]²=i(u,v) (3)

Then,

p(u,v)r(u,v)=\sqrt{[i(u,v)]} (4)

Because:

|FT[I(x,y)]|=i(u,v) (5),

the 2-dimensional light amount intensity distribution I (x,y) on the 2-dimensional detector to be measured is processed by Fourier transform. The i (u,v) is obtained by the equation (5). This is substituted in the equation (4), and optical transmission function p (u,v) r (u,v) of the optical system of the eye and the visual cell are calculated.

Hereunder, description will be given below on calculating procedure to estimate the visual acuity value of the eye under test based on the optical transmission function.

The optical transmission function p (u,v) r (u,v) obtained from calculation by the above equation relate to light amount intensity distribution of sinusoidal wave. Frequency characteristics of the eye to a sinusoidal wave chart is the so-called MTF. Using the optical transmission function as a mono-dimensional function, it is determined as:

MTF(u)=p(u)r(u) (6)

On the other hand, when the visual acuity value is to be judged, in case of a black-white Landolt ring used as a target for visual acuity test, for example, it is measured to which size of the Landolt ring the person under test can recognize the gap. The light amount intensity distribution of square wave corresponds to the size of the gap.

Here, the frequency characteristics (optical transmission function) of the eye to a square wave chart is called square-MTF (square frequency characteristics) (hereinafter referred as “S-MTF”). When visual acuity is measured, measurement is made according to the frequency characteristics with respect to the square wave chart, and the frequency characteristics of the eye to the sinusoidal wave chart (MTF) as given above must be converted to S-MTF.

S-MTF (u) as given above can be calculated from the following equation (7) based on the equation (6) as given above:

S-MTF(u)=4/π{MTF(u)−(MTF(3u))/3+(MTF(5u)) /5−(MTF(7u))/7+ . . . } (7)

The result of calculation by the equation (7) is indicated as S-MTF curve shown in FIG. 4. In the diagram, the value of S-MTF is represented on the axis of ordinate, and the frequency u is represented on the axis of abscissa.

When the visual acuity value of the person under test is estimated from the S-MTF curve, it is possible to determine a constant threshold value, and to estimate the visual acuity from an intersection of the threshold value and S-MTF curve. In case the person under test has small S-MTF value, the procedure may be likely to cause error in the estimated value. For this reason, in the present invention, the so-called modulation threshold (hereinafter referred as “MT (u)”), indicating the threshold value of a nervous system in the visual system, is used instead of a constant threshold value.

The value of MT (u) can be experimentally obtained by entering two light beams to the eye to directly form interference fringes on the retina of the fundus and by instructing a person to observe the condition of the interference fringes. The MT (u) thus obtained is sinusoidal wave MT (u). The value used as the threshold value is square-MT to square wave (hereinafter referred as “S-MT”), and this is converted from the sinusoidal wave MT (u).

The conversion can be made by the following equation similarly to the procedure to obtain S-MTF:

S-MT(u)=4/π{MT(u)−(MT(3u))/3+(MT(5u))/5−(MT(7u))/7+ . . . } (8)

The S-MT (u) obtained here indicates a boundary value, which can be identified by the eye under test. If the value is higher than S-MT (u), it can be identified by the eye under test. If the value is lower than S-MT (u), it cannot be identified by the eye under test.

Further, the S-MT (u) generally varies according to age of the person under test. There are prepared the function in the teen-agers defined as S-MT10 (u), that of the twenties defined as S-MT20 (u), and that of the thirties as S-MT30 (u) . . . Using the modulation-threshold (S-MTa (u)) corresponding to the age of the person under test, S-MTa(u) is superimposed on the graph shown in FIG. 4 which shows S-MTF of the person under test. Then, the frequency u corresponding to the intersection of S-MTF curve and S-MTa (u) curve is calculated.

For instance, in case the person under test is in the teen-age, the frequency u10 corresponding to the intersection of S-MT10 and S-MTF in FIG. 4 is obtained.

The relation between the so-called decimal visual acuity value (D.V.A.) and u is given by: D.V.A.=u/100. Based on the frequency u10 obtained above, the decimal visual acuity value (D.V.A.) can be obtained. Visual acuity value is obtained by using the threshold value, which corresponds to MT (u) of the age of the person under test, and the error can be reduced in the estimated value.

Thus, without relying on the answer from the person under test, the visual acuity value of the eye under test can be measured.

In the present embodiment, the position of the focusing lens 19 is regarded as the reference position, and the correction optical system 12 is set to correction amount 0, and the measurement is performed. Based on the result of the measurement, the visual acuity value of the naked eye of the person under test is estimated. The present invention is not limited to this case, and a visual acuity value after correcting the refraction of a certain amount can be also estimated if the correction optical system is adjusted or the focusing lens is moved, and measurement is made after correction of refraction of a certain amount and by performing similar calculation.

The eye's optical characteristic measuring system of the present invention comprises a target projecting system for projecting a target image on an ocular fundus of an eye under test, a photodetection optical system for guiding the target image to a photoelectric detector, a calculating unit for calculating an optical transmission function of the eye under test based on light amount intensity distribution of the target image detected by the photoelectric detector, and an arithmetic unit for calculating visual acuity of a person under test from intersection of the optical transmission function and a predetermined threshold value. As a result, visual acuity of an eye under test can be accurately estimated without relying on the so-called subjective optometric procedure, by which visual acuity is measured according to the answer from the person under test after showing various sizes of targets for visual acuity test. The measurement can be accomplished by simply projecting a predetermined target image to the ocular fundus of the eye under test and by measuring light amount intensity distribution of the target image.

What is claimed is:

1. An eye's optical characteristic measuring system, comprising a target projecting system for protecting a target image on an ocular fundus of an eye under test, a photodetection optical system for guiding the target image to a photoelectric detector, a calculating unit for calculating an optical transmission function of the eye under test based on light amount intensity distribution of the target image detected by said photoelectric detector, and an arithmetic unit for calculating visual acuity of a person under test from intersection of the optical transmission function and a predetermined threshold value.

2. An eye's optical characteristic measuring system according to claim 1, wherein said optical transmission function is square wave frequency characteristics.

3. An eye's optical characteristic measuring system according to claim 1, wherein said threshold value is a modulation threshold.

4. An eye's optical characteristic measuring system according to claim 3, wherein a plurality of said modulation threshold are prepared to correspond to age of the persons under test, and a modulation threshold suitable for each person under test is used.
